(19) United States
(12) Patent Application Publication
(10) Pub. No.: US 2015/0203471 A1
(43) Pub. Date: Jul. 23, 2015

(54) METHODS FOR IMPROVING CHICKEN PRODUCTION

(71) Applicant: ELI LILLY AND COMPANY, Indianapolis, IN (US)

(72) Inventors: Djamel Aissat, Fishers, IN (US); Brian D. Burke, New Palestine, IN (US); David Andrew Coates, Indianapolis, IN (US); Jeffrey Glen Sherman, Middleton, IN (US)

(73) Assignee: ELI LILLY AND COMPANY, Indianapolis, IN (US)

(21) Appl. No.: 14/419,791

(22) PCT Filed: Sep. 24, 2013
(86) PCT No.: PCT/US2013/061325

§ 371 Date: Feb. 5, 2015

Related U.S. Application Data
(60) Provisional application No. 61/708,838, filed on Oct. 2, 2012.

(51) Int. Cl. C07D 401/12; A23K 1/18; A23K 1/16; C07C 57/15

(52) U.S. Cl. CPC C07D 401/12; C07C 57/15; A23K 1/1826; A23K 1/1625

(57) ABSTRACT

The invention provided methods of improving the growth performance of and/or increasing the meat obtainable from chicken, and/or reducing gas emissions from a chicken or chicken raising operation, comprising administering to the chicken an effective amount of Compound 5 or a physiologically acceptable salt thereof. Also included in the invention are oral chicken feed compositions comprising an effective amount of the above compound, and the crystalline hemifumarate anhydrous salt Form II of the above compound.

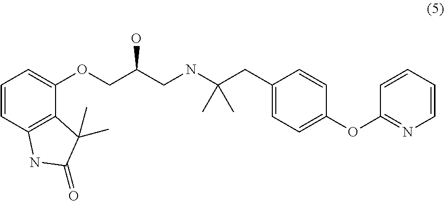 [SMILES: CC(C)(Cc1ccc(Oc2ccccn2)cc1)NC[C@H](O)COc1cccc2c1C(C)(C)C(=O)N2]

The raising of livestock animals is not only of great significance to the fields of animal health but also integral to the area of human meat consumption. An important goal in animal husbandry is to develop methods of utilizing biologically active agents which can improve the growth performance of the animal, as well as maintain/improve the quality of (meat to fat ratio, for example) and/or increase the quantity of the meat obtained from the animal. Ideally, such biologically active agents are effective to maintain/improve the quality of meat and to increase the quantity of meat.

In chicken raising operations, gasses are generated by the animal as well as when animal wastes break down. Particularly, the ammonia gas generated can be harmful to the health and growth rate of the chickens, and ammonia emissions to the atmosphere are an environmental concern. To address these problems, different methods have been used to control ammonia emissions from chicken raising operations. One method of controlling ammonia emissions is to apply a chemical to the animal waste. For example, in the production of broiler chickens it is known to apply a chemical such as sodium bisulfate, aluminum sulfate, iron sulfate or sulfuric acid to the litter on the floor of a chicken house. When ammonia from the chicken waste comes into contact with the chemical it reacts and is changed into solid ammonium sulfate and as a result it is not emitted into the air as gaseous ammonia. In a typical broiler chicken operation, the chemical is applied on the floor of the chicken house before the chickens are introduced into the house. The chickens cover the entire floor of the chicken house, and as they grow they become bigger and more packed together. After the chickens are in the house, there is currently no practical means of applying a chemical to the floor to control ammonia. Consequently, after the first two weeks of the grow out, ammonia emissions from the chicken waste resume and continue throughout the remainder of the grow out.

Accordingly, the present invention provides methods and compositions for improving the growth performance in chickens. Additionally, the present invention provides methods and compositions for increasing the quantity of meat obtainable from chickens. The invention also provides methods for reducing gas emissions from chickens and chicken raising operations. The compounds used in all embodiments of the present invention display favorable mutagenicity and/or teratogenicity characteristics.

The methods and compositions of the invention employ the following compound:

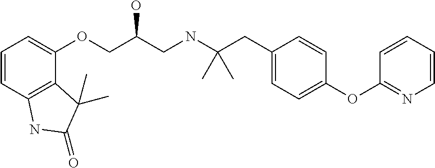 [SMILES: CC(C)(Cc1ccc(Oc2ccccn2)cc1)NC[C@H](O)COc1cccc2c1C(C)(C)C(=O)N2]

or a physiologically acceptable salt thereof.

The compounds of the invention may be made by processes known in the art. For example, the processes described in U.S. Pat. No. 6,534,504, WO2002/38544, WO200238543, U.S. Pat. No. 6,825,220, U.S. Pat. No. 7,122,680, U.S. Pat. No. 6,841,563, U.S. Pat. No. 6,093,735, U.S. Pat. No. 7,041,684, and WO2001/36412 illustrative processes which may be used to make the compounds of the invention.

The compounds of the present disclosure can react to form physiologically acceptable derivatives or salts that are also useful in the methods of this invention. The salts can be prepared using standard procedures for salt preparation. The term “physiologically acceptable salt” refers to an addition salt that exists in conjunction with the acidic or basic portion of the compound. Such salts include the physiologically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan.

For example, the compound can be neutralized with an appropriate acid to form an acid addition salt. The acid addition salts include salts formed by reaction with either an organic or inorganic acid such as, for example, sulfuric, hydrochloric, phosphoric, acetic, succinic, citric, lactic, maleic, fumaric, cholic, pamoic, mucic, glutamic, camphoric, glutaric, glycolic, phthalic, tartaric, formic, lauric, stearic, salicylic, methanesulfonic, benzenesulfonic, sorbic, picric, benzoic, cinnamic, and like acids. In one embodiment of the present invention, the physiologically acceptable salt of the compound is a hydrochloride salt. In one embodiment of the present invention, the physiologically acceptable salt of the compound is a hemifumarate salt, and particularly the crystalline hemifumarate anhydrous salt Form II, as described below.

The term “chicken” refers to the species Gallus gallus, including Gallus gallus domesticus. The chicken may be any age. Preferably, the chicken is less than 70 days old, less than 65 days old, or less than 60 days old. Preferably, the chicken is a broiler.

As used herein, “improving the growth performance” refers to enhancing, including reducing or increasing as the case may be, one or more aspects of the growth of a chicken. Such aspects include one or more of the following: increased average daily gain (ADG), increased gain efficiency, increased feed intake, increased hot carcass weight, increased average daily live weight gain, increased leg weight, increased breast weight, and decreased fat pad weight. “Increasing the quantity of meat’ obtainable from a chicken includes increasing the overall meat of a chicken, increasing the meat at the legs, and/or increasing the meat at the breast. “Reducing gas emissions” from a chicken or chicken raising operation refers to reducing one or both of NH₃ or H₂S from a chicken or chicken raising operation.

As used herein, the term “effective amount” refers to the quantity which, when administered to a chicken, improves the growth performance of, and/or increases the quantity of meat obtainable from, a chicken, and/or reduces gas emissions from a chicken or chicken raising operations. Preferably, the compounds of the invention are provided to the chickens in animal feed or water which the chickens may access at will. Additionally, the invention includes administering the compounds of the invention with other active ingredients. Therefore, “effective amount” includes amounts of compounds of the invention which, when combined with the administration of one or more other active ingredients, improves the growth performance of, and/or increases the quantity of meat obtainable from, a chicken, and/or reduces gas emissions from a chicken or chicken raising operation.

As used herein, the term “administering” refers to orally administering an effective amount of a compound of the present invention to a chicken. For oral administration, the compound of the present invention may be admixed with suitable carriers or diluents commonly employed in animal husbandry. Animal feedstuffs and feed comprising the compound of the present invention are provided as a further embodiment of the present invention. Typical carriers and diluents commonly employed in such feedstuffs include corn meal, corncob grits, soybean meal, alfalfa meal, rice hulls, soybean mill run, cottonseed oil meal, bone meal, ground corn, corncob meal, wheat middlings, limestone, dicalcium phosphate, sodium chloride, urea, distillers dried grain, vitamin and/or mineral mixes, cane molasses, water, or other liquid carriers and the like. Such carriers may promote a uniform distribution of the active ingredient. The compounds of the invention may be administered to a chicken of any age. Preferably, the compounds of the invention are administered to chickens up to 70, 65, or 60 days of age. In an oral animal feed composition, the amount of the compounds of the present invention may be from above 0 to 2 ppm, from 2 to 50 ppm, from 3 to 40 ppm, from 3.5 to 10 ppm, from 5 to 10 ppm, or about 7 ppm.

In some embodiments of the present invention, the administration of the compound to a chicken is for various durations of time. In one embodiment, the administration is for a short term of duration. As used herein, a “short term” of duration means daily administration of the compound for less than about 30 days, for example 14 days, 21 days, or 28 days. In another embodiment, the administration is for an intermediate term of duration. As used herein, an “intermediate term” of duration means daily administration of the compound for between about 31 days and about 60 days, for example 42 days, 48 days, or 54 days. In yet another embodiment, the administration is for a long term of duration. As used herein, a “long term” of duration means daily administration of the compound for greater than about 60 days, for example 75 days, 90 days, 100 days, 111 days, 120 days, or longer.

The following clauses illustrate the invention.
1. A method of improving the growth performance of a chicken, wherein said method comprises orally administering to said chicken an effective amount of the compound of the formula:

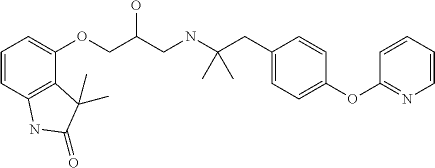 [SMILES: CC(C)(Cc1ccc(Oc2ccccn2)cc1)NCC(O)COc1cccc2c1C(C)(C)C(=O)N2]

or a physiologically acceptable salt thereof.
2. The method of clause 1, wherein the administration is effected by including the compound in an animal feed.
3. The method of clause 1 or clause 2, wherein the animal feed is provided to said chicken ad libitum.
4. The method of any one of clauses 1 to 3, wherein the physiologically acceptable salt of the compound is a hydrochloride or hemi-fumarate salt.
5. The method of any one of clauses 2 to 4, wherein the compound is present in the feed in an amount of between about 2 to about 50 ppm.
6. The method of any one of clauses 2 to 5, wherein said compound is present in the feed in an amount of between about 3.5 to about 10 ppm.
7. The method of any one of clauses 2 to 6, wherein said compound is present in the feed in an amount of at least about 7 ppm.
8. The method of any one of clauses 1 to 7, wherein the administration is for a short term of duration.
9. The method of any one of clauses 1 to 7, wherein the administration is for an intermediate term of duration.
10. The method of any one of clauses 1 to 7, wherein the administration is for a long term of duration.
11. The method of any one of clauses 1 to 10, wherein the chicken is Gallus gallus domesticus.
12. The method of any of clauses 1 to 11, wherein the chicken is less than 70 days old.
13. The method of any of clauses 1 to 12, wherein said chicken is male.
14. The method of any of clauses 1 to 13 wherein said chicken is a broiler.
15. The method of any of clauses 1 to 14, wherein said improved growth performance is at least one of average daily gain (ADG), gain efficiency, feed intake, hot carcass weight, average daily live weight gain, leg weight, breast weight, and fat pad weight.
16. A method of increasing the amount of meat of a chicken, wherein said method comprises orally administering to said chicken an effective amount of the compound of the formula:

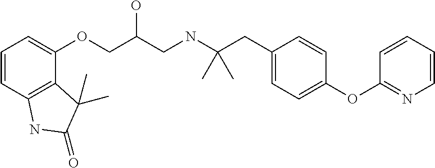 [SMILES: CC(C)(Cc1ccc(Oc2ccccn2)cc1)NCC(O)COc1cccc2c1C(C)(C)C(=O)N2]

or a physiologically acceptable salt thereof.
17. The method of clause 16, wherein the administration is effected by including the compound in an animal feed.
18. The method of clause 16 or clause 17, wherein the animal feed is provided to said chicken ad libitum.
19. The method of any one of clauses 16 to 18, wherein the physiologically acceptable salt of the compound is a hydrochloride or hemi-fumarate salt.
20. The method of any one of clauses 19 to 21, wherein the compound is present in the feed in an amount of between about 2 to about 50 ppm.
21. The method of any one of clauses 17 to 20, wherein said compound is present in the feed in an amount of between about 3.5 to about 10 ppm.
22. The method of any one of clauses 17 to 21, wherein said compound is present in the feed in an amount of at least about 7 ppm.
23. The method of any one of clauses 16 to 22, wherein the administration is for a short term of duration.
24. The method of any one of clauses 16 to 22, wherein the administration is for an intermediate term of duration.
25. The method of any one of clauses 16 to 22, wherein the administration is for a long term of duration.
26. The method of any one of clauses 16 to 25, wherein the chicken is Gallus gallus domesticus.
27. The method of any of clauses 16 to 29, wherein the chicken is less than 70 days old.
28. The method of any of clauses 16 to 27, wherein said chicken is male.
29. The method of any of clauses 16 to 28 wherein said chicken is a broiler
30. The method of any of clauses 16 to 29, wherein said increase is an increase one or more of the overall meat, the meat at the legs, and/or the meat at the breast.
31. A method of decreasing gas emissions from a chicken or a chicken raising operation, wherein said method comprises orally administering to said chicken an effective amount of the compound of the formula:

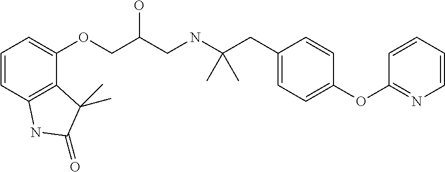 [SMILES: CC(C)(Cc1ccc(Oc2ccccn2)cc1)NCC(O)COc1cccc2c1C(C)(C)C(=O)N2]

or a physiologically acceptable salt thereof.
32. The method of clause 31 wherein the administration is effected by including the compound in an animal feed.
33. The method of clause 31 or clause 32, wherein the animal feed is provided to said chicken ad libitum.
34. The method of any one of clauses 31 to 33, wherein the physiologically acceptable salt of the compound is a hydrochloride or hemi-fumarate salt.
35. The method of any one of clauses 31 to 34, wherein the compound is present in the feed in an amount of between about 2 to about 50 ppm.
36. The method of any one of clauses 31 to 35, wherein said compound is present in the feed in an amount of between about 3.5 to about 10 ppm.
37. The method of any one of clauses 31 to 36, wherein said compound is present in the feed in an amount of at least about 7 ppm.
38. The method of any one of clauses 31 to 37, wherein the administration is for a short term of duration.
39. The method of any one of clauses 31 to 37, wherein the administration is for an intermediate term of duration.
40. The method of any one of clauses 31 to 37, wherein the administration is for a long term of duration.
41. The method of any one of clauses 31 to 40, wherein the chicken is Gallus gallus domesticus.
42. The method of any of clauses 31 to 40, wherein the chicken is less than 70 days old.
43. The method of any of clauses 31 to 41, wherein said chicken is male.
44. The method of any of clauses 31 to 42, wherein said chicken is a broiler
45. The method of any of clauses 31 to 43, wherein said reduction is in the amount of NH₃.
46. The methods of any of clauses 1 to 45 wherein an additional active ingredient is administered to said chicken.
47. An oral chicken feed composition comprising an effective amount of a compound of the formula:

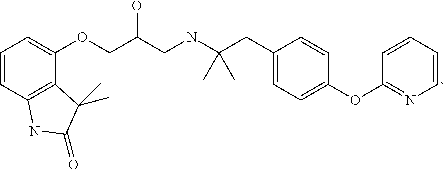 [SMILES: CC(C)(Cc1ccc(Oc2ccccn2)cc1)NCC(O)COc1cccc2c1C(C)(C)C(=O)N2]

or a physiologically acceptable salt thereof, in association with a suitable carrier therefor, and optionally one or more active ingredients.
48. The composition of clause 47, wherein the physiologically acceptable salt of the compound is a hydrochloride or hemi-fumarate salt.
49. The composition of clause 47 or clause 48, wherein the compound is present in the feed in an amount of between about 2 to about 50 ppm.
50. The composition of any one of clauses 47 to 49, wherein said compound is present in the feed in an amount of between about 3.5 to about 10 ppm.
51. The composition of any one of clauses 47 to 50, wherein said compound is present in the feed in an amount of at least about 7 ppm.
52. The methods or compositions of any of clauses 1-51, wherein said compound is

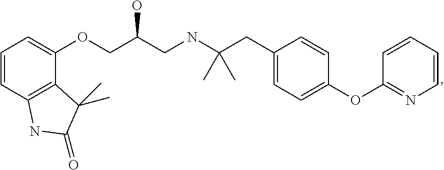 [SMILES: CC(C)(Cc1ccc(Oc2ccccn2)cc1)NC[C@H](O)COc1cccc2c1C(C)(C)C(=O)N2]

or a physiologically acceptable salt thereof

Study 1

The following compounds are used in the study.

### Compound 1: Positive Control

### 2-[4-(2-{[(2S)-2-hydroxy-3-(2-thiophen-2-ylphenoxy)propyl]amino}-2-methylpropyl)phenoxy]pyridine-3-carbonitrile

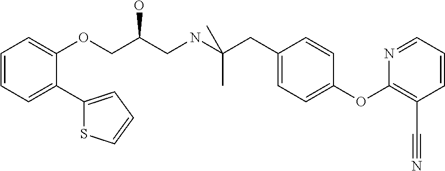 [SMILES: CC(C)(Cc1ccc(Oc2ncccc2C#N)cc1)NC[C@H](O)COc1ccccc1-c1cccs1]

### Compound 2

### (2S)-1-{[1,1-dimethyl-2-(4-{[5-(morpholin-4-ylcarbonyl)pyridin-2-yl]oxy}phenyl)ethyl]amino}-3-(1H-indazol-4-yloxy)propan-2-ol

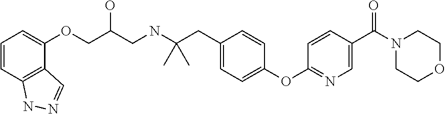 [SMILES: CC(C)(Cc1ccc(Oc2ccc(C(=O)N3CCOCC3)cn2)cc1)NCC(O)COc1cccc2[nH]ncc12]

### Compound 3

### 4-({(2S)-3-[(1,1-dimethyl-2-{4-[4-(methylsulfonyl)phenoxy]phenyl}ethyl)amino]-2-hydroxypropyl}oxy)-1,3-dihydro-2H-benzimidazole-2-thione

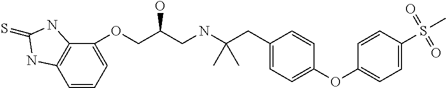 [SMILES: CC(C)(Cc1ccc(Oc2ccc(S(C)(=O)=O)cc2)cc1)NC[C@H](O)COc1cccc2[nH]c(=S)[nH]c12]

### Compound 4

### 2-{4-[2-({(2S)-2-hydroxy-3-[2-(1H-indol-2-yl)phenoxy]propyl}amino)-2-methylpropyl]phenoxy}pyridine-3-carboxamide

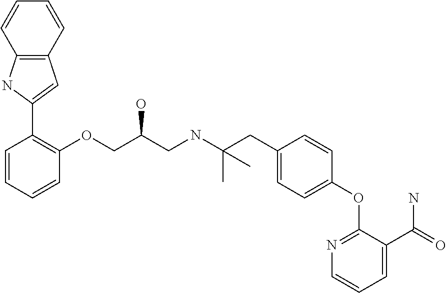 [SMILES: CC(C)(Cc1ccc(Oc2ncccc2C(N)=O)cc1)NC[C@H](O)COc1ccccc1-c1cc2ccccc2[nH]1]

### Compound 5

### 4-[{(2S)-3-({1,1-dimethyl-2-[4-(pyridin-2-yloxy)phenyl]ethyl}amino)-2-hydroxypropyl]oxy}-3,3-dimethyl-1,3-dihydro-2H-indol-2-one hydrochloride

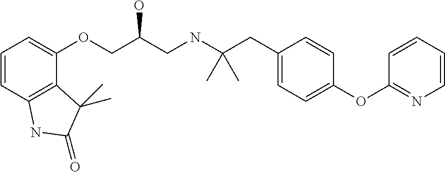 [SMILES: CC(C)(Cc1ccc(Oc2ccccn2)cc1)NC[C@H](O)COc1cccc2c1C(C)(C)C(=O)N2]

### Compound 6

### (2S)-1-{[1,1-dimethyl-2-(4-{[5-(morpholin-4-ylcarbonyl)pyridin-2-yl]oxy}phenyl)ethyl]amino}-3-(1H-indol-4-yloxy)propan-2-ol

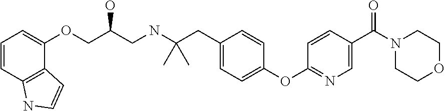 [SMILES: CC(C)(Cc1ccc(Oc2ccc(C(=O)N3CCOCC3)cn2)cc1)NC[C@H](O)COc1cccc2[nH]ccc12]

### Compound 7

### 4-({(2S)-3-[(2-{4-[2,4-bis(methylsulfonyl)phenoxy]phenyl}-1,1-dimethylethyl)amino]-2-hydroxypropyl}oxy)-1H-indole-2-carboxamide

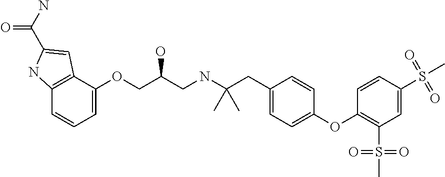 [SMILES: CC(C)(Cc1ccc(Oc2ccc(S(C)(=O)=O)cc2S(C)(=O)=O)cc1)NC[C@H](O)COc1cccc2[nH]c(C(N)=O)cc12]

### Compound 8

### 4-[4-(2-{[2-hydroxy-3-(3-hydroxyphenoxy)propyl]amino}-2-methylpropyl)phenoxy]benzoic acid dihydrochloride

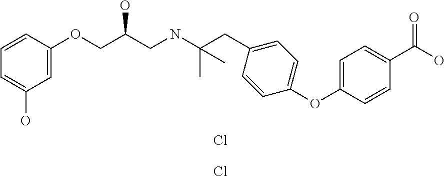 [SMILES: CC(C)(Cc1ccc(Oc2ccc(C(=O)O)cc2)cc1)NC[C@H](O)COc1cccc(O)c1 ; Cl ; Cl]

### Compound 9

### 2-[4-(2-{[(2S)-2-hydroxy-3-(1H-indazol-4-yloxy)propyl]amino}-2-methylpropyl)phenoxy]pyridine-3-carboxamide

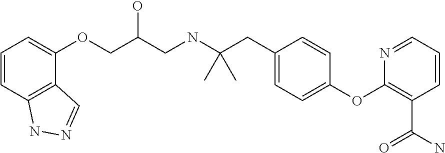 [SMILES: CC(C)(Cc1ccc(Oc2ncccc2C(N)=O)cc1)NCC(O)COc1cccc2[nH]ncc12]

An objective of this study is to evaluate growth performance and carcass responses of male broilers by comparing non-zero dosages of the above compounds versus a positive control (Compound 1) and negative controls. Table 1 sets out the specific treatment groups:

TABLE 1

| | | Trt. Phase | | | |
|---|---|---|---|---|---|
| Treatment | Trt. Dose | Duration | No. of | Animals | Total No. |
| Description | (ppm) | (days) | Pens | per pen | of Animals |
| Control | 0 | 14 | 10 | 14 | 140 |
| Positive | 15 | 14 | 10 | 14 | 140 |
| Control | | | | | |
| Compound 1 | | | | | |
| Compound 2 | 15 | 14 | 10 | 14 | 140 |
| Compound 3 | 15 | 14 | 10 | 14 | 140 |
| Compound 4 | 15 | 14 | 10 | 14 | 140 |
| Compound 5 | 15 | 14 | 10 | 14 | 140 |
| Compound 6 | 15 | 14 | 10 | 14 | 140 |
| Compound 7 | 15 | 14 | 10 | 14 | 140 |
| Compound 8 | 15 | 14 | 10 | 14 | 140 |
| Compound 9 | 15 | 14 | 10 | 14 | 140 |
| Total | | | 100 | | 1400 |

The route of administration is oral, with the compounds mixed in feed, and provided Ad libitum. The age of the chickens of Day 1 of the study is 35 days old. Body weights are taken on the following days:
1) Study Day 1 (pen weight)
2) Study Day 14 (individual animal weights)
3) Study Day 15 (individual weights on selected animals)
4) Study Day 16 (blocks 1-5)
5) Study Day 17 (blocks 6-10).

Following collection of final weights, the following processing data is collected (hot weights) on individual animals:
1) Live weight
2) Hot carcass weight
3) Fat pad weight
4) Hot breast muscle weight
5) Hot bone in leg weight

The positive control, Compound 1, results in improved ADG, feed intake, average daily live weight gain only. All of the compounds result in a numerical improvement in hot carcass weight. Three compounds, Compounds 2, 5, and 9, display significantly better improvement than both the negative control and the positive control. Gain is associated with a significant increase in feed intake for all three compounds and is significantly greater than untreated control for Compounds 2, 5, and 9. Compounds 2, 5, and 9 also reduce fat pad weight but the difference is significant only for Compound 5 and 9. Percent change from control is summarized in Table 2 below for compounds that perform better than the untreated control.

TABLE 2
Compounds significantly better at improving performance than positive control
(Compound 1) versus negative control based on percent change from control

| | | | | Hot | Ave daily | | | Fat |
|---|---|---|---|---|---|---|---|---|
| | | Gain | Feed | carcass | live wt | Leg | Breast | Pad |
| | ADG % | effic % | intake % | wt % | gain % | wt % | wt % | wt % |
| Compound # | inc. | inc. | inc. | inc. | inc. | inc. | inc. | dec. |
| Compound 1 | 4.4 | 1.6* | 2.7 | 2.4* | 4.0 | 1.2* | 3.5* | 9.8* |
| Compound 2 | 8.7 | 3.8 | 4.8 | 6.1 | 8.3 | 8.2 | 6.1 | 12.8* |
| Compound 3 | 1.6* | | | 2.7 | | | | |
| Compound 4 | −1.6* | | | 0.3* | | | | |
| Compound 5 | 5.1 | 4.9 | 1.1* | 5.8 | 5.8 | 8.1 | 6.0 | 17.4 |
| Compound 6 | 3.9 | | | 3.8 | | | | |
| Compound 7 | 2.0* | | | 1.2* | | | | |
| Compound 8 | −0.4* | | | 0.8* | | | | |
| Compound 9 | 8.9 | 6.7 | 2.1* | 5.0 | 9.3 | 7.9 | 4.8 | 17.8 |

*Not significantly increased or decreased from negative control

Study 2

Compounds 2, 5, and 9 are used in this study. The study is conducted to evaluate growth performance and carcass responses of male broilers by comparing non-zero dosages of the compounds, versus negative control, administered in the last 14 days prior to slaughter. Table 3 sets out the specific treatment groups.

TABLE 3

| | | Trt. Phase | | | |
|---|---|---|---|---|---|
| Treatment | Trt. Dose | Duration | No. of | Animals | Total # |
| Description | (ppm) | (days) | Pens | per pen | Animals |
| GP 1: Control | 0 | 14 | 16 | 14 | 224 |
| GP 2: Compound 9 | 3.5 | 14 | 16 | 14 | 224 |
| GP 3: Compound 9 | 7 | 14 | 16 | 14 | 224 |
| GP 4: Compound 2 | 3.5 | 14 | 16 | 14 | 224 |
| GP 5: Compound 2 | 7 | 14 | 16 | 14 | 224 |
| GP 6: Compound 5 | 3.5 | 14 | 16 | 14 | 224 |
| GP 7: Compound 5 | 7 | 14 | 16 | 14 | 224 |
| Total | | | 112 | | 1568 |

The route of administration is oral, with the compounds mixed in feed, and provided Ad libitum. The age of the chickens of Day 1 of the study is 35 days old. Body weights are taken on the following days:
1) Study Day 1 (pen weight)
2) Study Day 15 (individual animal weights)
3) Study Day 16 (individual weights on selected animals in animals in blocks 1-4)
4) Study Day 17 (individual weights on selected animals in animals in blocks 5-8)

Following collection of final weights, the following processing data is collected (hot weights) on individual animals:
1) Live weight
2) Hot carcass weight
3) Fat pad weight
4) Hot breast muscle weight
5) Hot bone in leg weight

The treatment effect is significant when comparing the negative control group (GP1) to all treatment groups for ADG, G/F and F/G (gain:feed efficiency and feed:gain efficiency, respectively), Hot Carcass Weight, Hot Breast Weight, and Hot Bone-In Leg Weight. Additionally, animals in groups GP3, GP6, and GP7 have significantly higher ADG's than those in the negative control group Animals in group GP3 have significantly higher ADFIs than those in the negative control group. Animals in group GP4 have significantly lower ADFI's than those in the negative control group Animals in groups GP2, GP3, GP5, GP6, and GP7 have significantly higher G/F ratios, hot carcass weights, hot breast muscle weights, and hot bone-in leg weights, than those in the negative control group. Animals in groups GP2, GP3, GP5, GP6, and GP7 have significantly lower F/G ratios than those in the negative control group.

Study 3

This study is conducted to determine the dose response of the Compound 5 measured in live performance and carcass yields of commercial broilers raised to a small-size market when fed a “typical” feed. 2400 Hubbard M99 X Cobb chicks (1200 male/1200 female) are used in this study. Compound 5 is administered at the following doses: 0, 2, 4, 8, and 16 ppm, orally in feed, beginning when the chicks are 24 days of age and ending when the chicks are 38 days of age. The five different Compound 5 doses are administered in a mash diet formulated to meet all nutrient recommendations using only commercially available ingredients.

Parameters measured in the study include ADG (average daily gain), ADFI (average daily feed intake), GE (gain efficiency), and FE (feed efficiency). The birds are measured for carcass (without giblets), breast muscle (boneless-skinless), and leg-quarter weights. The live body weight on the day of processing is used to calculate the yields for carcass, breast meat, and leg quarters for the birds.

Improvements are observed for GE, FE, and leg-quarter weight and yield, although doses at above 2 ppm do not show additional benefit. GE and FE are positively impacted only in female broilers. Compound 5 responses may be linked to age due to fat deposit status, and a 24-38 day-old broiler may not have shown an impact on other measured parameters due to the young age of the animal, and therefore lack of lipid tissue deposition. Additionally, the ability of the females, but not the males, in this study, to improve both FE and GE further suggests that these responses may be associated with fat body composition considering that females accrue lipid tissue quicker and at higher proportions than male broilers.

Study 4

This study is conducted to investigate the effects of Compound 5 on live performance and carcass traits of broilers, and to evaluate the emission of various gases from broilers provision of feed with or without Compound 5. Ross X Ross 708 male chicks are used in this study. Compound 5 is administered at the following doses: 0 and 16 ppm, orally in feed, beginning when the chicks are 34 days of age and ending when the chicks are 48 days of age. The Compound 5 doses are administered in a mash diet formulated to meet all nutrient recommendations using only commercially available ingredients.

Parameters measured in the study include ADG, ADFI, and FCR. The birds are measured for carcass (without giblets), breast muscle (boneless-skinless), leg-quarter, and abdominal fat weights. The live body weight on the day of processing is used to calculate the yields for carcass, breast meat, and leg quarters for the birds. Also measured are emissions of various gases. Cumulative gas emission are monitored and calculated for the following gases: NH₃, NO, NO₂, H₂S, SO₂, methane/non-methane total hydrocarbon, O₂, and CO₂. The efficacy of Compound 5 is determined by the cumulative emission of these gases, as well as by standardizing these cumulative values to their body, carcass, breast meat, and leg-quarter weights.

Supplementation with Compound 5 results in improvements in all live performance carcass traits monitored, except for ADFI and mortality. The cumulative emission of NH₃ and H₂S from Compound 5 dosed birds is reduced by 29.2 and 19.0%, respectively, when compared to untreated birds. When taking into account the increase in live weight of the birds or its saleable cuts, the resultant standardized differences increase to more than 30% reduction in NH₃ emission.

Toxicity Screening

Compound 5 displays favorable mutagenicity and/or teratogenicity characteristics. For instance, Compound 5 shows better results in the Ames assay (mutagenicity) and the Zebrafish assay (teratogenesis/chromosomal impact) than the other compounds tested.

Preparation of Crystalline Hemifumarate Anhydrous Salt Form II of Compound 5

Reproducibility issues of acceptable crystal forms of the HCl salt of Compound 5 has led to the search for a more suitable salt form. The crystalline hemifumarate anhydrous salt Form II of Compound 5 displays favorable crystallinity, yield, and reproducibility. The crystalline hemi-fumarate anhydrous salt form of Compound 5 is one of two forms of Compound 5 hemi-fumarate salt—the other is a solvated form which has unfavorable characteristics. Other salt forms of Compound 5 also have unfavorable characteristics, such that the hemi-fumarate anhydrous salt form 2 of compound is the only crystalline salt form determined to meet the necessary standards.

Crystalline hemifumarate anhydrous salt Form II is prepared by placing 2 g of the free base of Compound 5 in a vial. The solid is dissolved in 10 mL of EtOH while stiffing at 1000 rpm at 60° C., to give a clear yellow solution. This solution is then slowly mixed with 251 mg of fumaric acid (dissolved in 5 mL EtOH) in 1 mL aliquots. The sample is stirred for 15 minutes, and a very thick white slurry results. Another 15 mL of EtOH is added to bring the total solvent volume to 30 mL. The sample is slurried for another 30 minutes at 60° C. A thick slurry of white solid results. The white solid is isolated by vacuum filtration and dried in place under vacuum and air stream for 10 minutes. The source vial and cake is washed 2 times with 10 mL aliquots of ethanol to maximize recovery. A thick cake of white solid results, and this solid is placed in the 65° C. vacuum oven overnight. 1.878 g is recovered (75.48% yield).

Differential scanning calorimetry analyses are carried out on a TA Instruments DSC unit Q2000. Samples are heated in crimped aluminum pans from 25 to 300° C. at 10° C./min with a nitrogen purge of 50 mL/min. The DSC temperature is calibrated with indium standard, onset of 156.3-156.9° C. Crystalline hemifumarate anhydrous salt Form II of Compound 5 displays a melting point onset at 203.73° C. by DSC.

The X-ray diffraction (XRD) patterns of crystalline solids are obtained on a Bruker D4 Endeavor X-ray powder diffractometer, equipped with a CuKa source λ=1.54060 Å) and a Vantec detector, operating at 35 kV and 50 mA. The sample is scanned between 4 and 40° in 2θ, with a step size of 0.009° in 20 and a scan rate of 0.5 seconds/step, and with 0.6 mm divergence, 5.28 fixed anti-scatter, and 9.5 mm detector slits. The dry powder is packed on a quartz sample holder and a smooth surface is obtained using a glass slide. The crystal form diffraction patterns are collected at ambient temperature and relative humidity. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. Furthermore, it is also well known in the crystallography art that for any given crystal form the angular peak positions may vary slightly. For example, peak positions can shift due to a variation in the temperature or humidity at which a sample is analyzed, sample displacement, or the presence or absence of an internal standard. In the present case, a peak position variability of 0.2 in 2θ will take into account these potential variations without hindering the unequivocal identification of the indicated crystal form. Confirmation of a crystal form may be made based on any unique combination of distinguishing peaks (in units of ° 2θ), typically the more prominent peaks. (United States Pharmacopeia #35, National Formulary #30, Chapter <941>, pages 427-432, 2012). The crystal form diffraction patterns, collected at ambient temperature and relative humidity, are adjusted based on NBS standard reference material 675 (mica) with peaks at 8.853 and 26.774 degrees 2-theta.

A prepared sample of the crystalline hemifumurate anhydrous salt Form II is characterized by an X-ray diffraction pattern using CuKa radiation as having diffraction peaks (2-theta values) as described in Table 4 below, and in particular having peaks at 19.32 in combination with one or more of the peaks selected from the group consisting of 12.32, 14.69, and 21.09; with a tolerance for the diffraction angles of 0.2 degrees.

TABLE 4

Compound 5 Crystalline Hemifumarate
Anhydrous Salt Form II Peak Positions

| | Angle (°2- | Relative Intensity |
|---|---|---|
| Peak | Theta) +/− 0.2° | (% of most intense peak) |
| 1 | 5.22 | 34.70 |
| 2 | 6.74 | 38.30 |
| 3 | 9.85 | 37.50 |
| 4 | 12.32 | 94.30 |
| 5 | 13.27 | 34.40 |
| 6 | 14.69 | 95.30 |
| 7 | 16.46 | 35.90 |
| 8 | 19.32 | 100.00 |
| 9 | 21.09 | 72.00 |
| 10 | 26.54 | 51.30 |

1-16. (canceled)

17. A method of improving the growth performance of a chicken, increasing the quantity of meat of a chicken, and/or reducing gas emissions from a chicken or a chicken raising operation, wherein said method comprises orally administering to said chicken an effective amount of the compound of the formula: 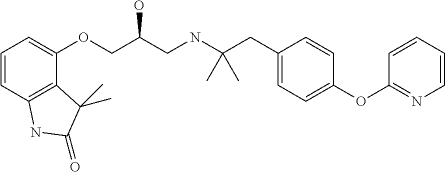 [SMILES: CC(C)(Cc1ccc(Oc2ccccn2)cc1)NC[C@H](O)COc1cccc2c1C(C)(C)C(=O)N2]
or a physiologically acceptable salt thereof.

18. The method of claim 17, wherein the physiologically acceptable salt of the compound is a hydrochloride or hemi-fumarate salt.

19. The method of claim 17, wherein the administration is effected by including the compound in an animal feed.

20. The method of claim 19, wherein the compound is present in the feed in an amount of between about above 0 to about 50 ppm.

21. The method of claim 19, wherein said compound is present in the feed in an amount of at least about 7 ppm.

22. The method of claim 17, wherein the administration is for a short term of duration.

23. The method of claim 17, wherein the chicken is Gallus gallus domesticus.

24. The method of claim 17, wherein said reduction is in the amount of NH₃.

25. The method of claim 17, wherein an additional active ingredient is administered to said chicken.

26. An oral chicken feed composition comprising an effective amount of a compound of the formula: 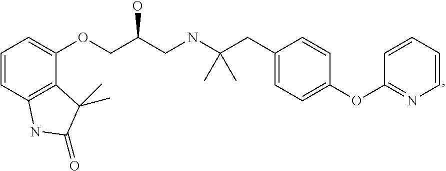 [SMILES: CC(C)(Cc1ccc(Oc2ccccn2)cc1)NC[C@H](O)COc1cccc2c1C(C)(C)C(=O)N2]
or a physiologically acceptable salt thereof, in association with a suitable carrier therefor, and optionally one or more active ingredients.

27. The composition of claim 26, wherein the physiologically acceptable salt of the compound is a hydrochloride or hemi-fumarate salt.

28. The composition of claim 26, wherein the compound is present in the feed in an amount of between about above 0 to about 50 ppm.

29. The composition of claim 26, wherein said compound is present in the feed in an amount of at least about 7 ppm.

30. The crystalline hemifumarate anhydrous salt Form II of the compound of the formula 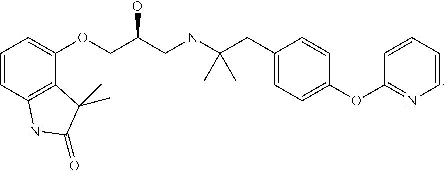 [SMILES: CC(C)(Cc1ccc(Oc2ccccn2)cc1)NC[C@H](O)COc1cccc2c1C(C)(C)C(=O)N2]
